(19) United States
(12) Patent Application Publication
(10) Pub. No.: US 2008/0109038 A1
(43) Pub. Date: May 8, 2008

(54) Threaded suture anchor and inserter device

(75) Inventors: Anton J. Steiner, Wharton, NJ (US); David I. Knight, Hopelawn, NJ (US); Cesar D. Chavista Para, Jersey City, NJ (US); James Shock, Glen Rock, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/594,208

(22) Filed: Nov. 8, 2006

(51) Int. Cl. A61B 17/04

(52) U.S. Cl. 606/232

(57) ABSTRACT

A sterile suture anchor and drive kit with the suture anchor having a cylindrical body portion with a generally tapered distal end portion and a screw thread extending along a cylindrical body portion. A plurality of parallel longitudinal grooves are cut into the cylindrical body interrupting opposed portions of the screw thread and a connecting groove is cut through the distal end portion of the suture anchor engaging the plurality of longitudinal grooves. A closed suture loop is mounted in the longitudinal grooves and the connecting groove. A driver is adapted to be mounted to the suture anchor, the driver comprising a handle with a tube mounted to the handle, the tube being provided with outwardly extending drive tip members adapted to be inserted into the suture anchor longitudinal grooves to engage the groove side walls of the suture anchor to apply driving torque to the suture anchor.

### RELATED APPLICATIONS

This are no related applications.

### STATEMENT REGARDING FEDERALLY SPONSORED RESEARCH OR DEVELOPMENT

Not applicable.

### REFERENCE TO SEQUENCE LISTING. A TABLE OR A COMPUTER PROGRAM LISTING COMPACT DISC APPENDIX

None.

### BACKGROUND OF THE INVENTION

1. Field of Invention

The field of art to which this invention relates is generally directed to suture anchors and more specifically to threaded suture anchors constructed of allograft bone which hold a suture.

2. Description of the Prior Art

As the treatment of injuries to joints and soft tissue has progressed, a need has developed for medical devices which can be used to attach tendons, ligaments and other soft tissue to bone. When surgically repairing an injured joint, it is preferable to restore the joint by reattaching the damaged soft tissues such as ligaments and tendons to bone rather than replacing them with an artificial material.

An increase in the incidence of injuries to joints involving soft tissue has been observed. This increased incidence of injuries may be due, at least in part, to an increase in participation by the public in various physical activities such as sports and other recreational activities. These types of activities increase the loads and stress placed uponjoints, sometimes resulting injoint injuries with corresponding damage to associated soft tissue. There are well over 500,000 surgical procedures performed in the United States annually in which soft tissue was reattached to a bone in various joints including the shoulder, hip and knee.

One conventional orthopedic procedure for reattaching soft tissue to bone is performed by initially drilling or punching holes or tunnels at predetermined locations through a bone in the vicinity of a joint. The surgeon approximates soft tissue to the surface of the bone using sutures threaded through these holes or tunnels. This method is a time consuming procedure resulting in the generation of numerous bone tunnels. A known complication of drilling tunnels across bone is that nerves and other soft tissue may be injured by the drill bit or orthopaedic pin as it exits the far side of the bone. Also, it may be anatomically challenging to reach and/or secure a suture that has been passed through a tunnel. When securing the suture or wire on the far side of the bone, nerves and soft tissues can become impinged and damaged.

Screws are also used to secure soft tissues adjacent to the bone surface. Screws suffer from a disadvantage in that they may loosen with time, thereby requiring a second operation to remove the loosened screw. In addition, when the screws are set in bone, the heads of the screws frequently protrude above the surface of the bone in which they are set, thereby presenting an abrasive surface which may create wear problems with surrounding tissue. Once a hole has been made in the bone it may be impossible to relocate the hole at a small distance away from its original position due to the disruption of the bone structure created by the initial hole. Finally, the nature of a screw attachment tends to require a flat attachment geometry; the pilot hole must generally be located on a relatively flat section of the bone, and toothed washers must frequently be used in conjunction with the screws to fasten the desired tissue to the target bone. As a result of these constraints, it may be necessary to locate the attachment point at less than an optimal position.

In order to overcome a number of the problems associated with the use of the conventional soft tissue to bone attachment procedures, suture anchors have been developed and are now frequently used to attach soft tissue to bone. A suture anchor, commonly referred to as a bone anchor, is an orthopedic, medical device which is typically implanted into a cavity drilled or punched into a bone. The bone cavity is typically referred to as a bore hole and if it does not extend through the bone is typically referred to as a “blind hole”. The bore hole is typically drilled or punched through the outer cortical layer of the bone and into the inner cancellous layer. The suture anchor may be engaged in the bore hole by a variety of mechanisms including friction fit, barbs which are forced into the cancellous layer of bone or by threading into pre-threaded bores in the bone mass or using self tapping threads. Suture anchors have many advantages including reduced bone trauma, simplified application procedures, and decreased likelihood of suture failure. Suture anchors may be used in shoulder reconstruction for repairing the glenohumeral ligament and may also be used in surgical procedures involving rotator cuff repair, ankle and wrist repair, bladder neck suspension, and hip replacement.

Suture anchors typically have a hole or eyelet for receiving a suture. The suture extends out from the bore hole and is used to attach soft tissue. The suture anchors presently described in the art may be made of absorbable materials which absorb over time, or they may be made from various non-absorbable, biocompatible materials. Although most suture anchors described in the art are made from non-absorbable materials, the use of absorbable suture anchors may result in fewer complications since the suture anchor is absorbed and replaced by bone over time. The use of absorbable suture anchors may also reduce the likelihood of damage to local joints caused by anchor migration. Moreover, when an absorbable suture anchor is fully absorbed it will no longer be present as a foreign body. It is also advantageous to construct the bone anchor out of allograft cortical bone as this material will result in natural filling in of the bore with bone in the original bone base and the elimination of foreign material from the site. Allograft bone anchors are also radiopaque for visualization under x-ray imaging.

It is also a problem that most of the bone anchors currently used are prepacked with sutures attached in kit form forcing the surgeon to use a specific type of suture and the hospital to carry large numbers of bone anchors in inventory with varying suture sizes.

The use of threaded bone anchors is well known as is shown in U.S. Pat. Nos. 5,814,070; 5,851,219; 5,899,920; 5,904,704; 6,096,060; 6,139,565; 6,231,606; 6,264,677 and 6,267,766.

A number of prior art patents such as U.S. Pat. Nos. 6,508,830; 5,941,882 and 5,733,307 are directed toward threaded bone anchors which have driver positioning grooves or troughs cut longitudinally along the anchor body intersecting the threads to receive sutures during the bone anchor insertion process.

U.S. Pat. No. 5,824,011 is directed toward a threaded bone anchor with a suture receiving eyelet. The anchor body has channels cut into its sides to receive driver torque applicators. The anchor is provided with a male member having a suture receiving eyelet, the male member fitting into a same shaped female configuration in the driver head.

U.S. Pat. No. 6,111,164 shows a bone insert which is formed from human cortical bone which is adapted to be driven into bone and the aforementioned U.S. Pat. No. 6,508,830 shows a threaded allograft bone anchor which can be mounted into the bone.

Although suture anchors for attaching soft tissue to bone are available for use by the orthopedic surgeon, there is a need in this art for novel suture anchors having improved performance characteristics, such as ease of insertion and greater resistance to “pull-out”.

### SUMMARY OF THE INVENTION

The present invention is directed toward a suture anchor constructed of allograft human bone which is threaded and has a plurality of longitudinal grooves cut into its outer surface intersecting the helical thread to hold the drive elements of a driver instrument and an upper recess connecting the longitudinal grooves to hold a suture loop. The distal end of the suture anchor is cut at a 45° angle with a flat end surface transverse to the central longitudinal axis of the bone anchor body.

The present invention provides a technical advantage in that it provides a channel in the suture anchor in which a suture loop resides during insertion of the bone anchor into the bone while also allowing the driver to apply torque along the sides of the bone anchor so that the anchor is less susceptible to mechanical breakage.

It is thus an object of the present invention to provide a suture anchor which can be used with a wide variety of sutures from different manufacturers allowing the surgeon the choice of sutures and suture composition, thus saving the hospital from stocking a large number of prepacked bone anchors and suture kits.

Therefore, it is another object of the present invention to provide a suture anchor which is simple to apply and is mechanically stable when implanted in bone.

It is a further object of the present invention to provide an absorbable suture anchor made of cortical bone.

Accordingly, one of the objects of the present invention is to provide an allograft suture anchor which promotes the use of natural bone growth in the bone bore hole.

Another object of the present invention is to provide a novel suture anchor with a preformed suture loop which can hold the suture outside the bone so that the free end of the suture can then be used to attach the desired object (e.g. a ligament or prosthesis) to the bone.

And another object of the present invention is to provide a novel suture anchor for anchoring one end of a piece of conventional suture in bone which has high tissue acceptability, prevents back out and is reliable in use.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a perspective view of the inventive bone suture anchor;

FIG. 2 is a perspective view of the inventive suture anchor of FIG. 1 with suture loop and suture;

FIG. 3 is an enlarged side elevational view of the suture anchor shown in FIG. 1;

FIG. 4 is a cross sectional view of the bone suture anchor of FIG. 3 taken along line 4′-4′ with the mounted suture loop shown in FIG. 2 shown in phantom;

FIG. 5 is a front elevational view of the suture anchor shown in FIG. 3;

FIG. 6 is a cross sectional view of the suture anchor of FIG. 5 taken along lines 6′-6′;

FIG. 7 is a perspective view of the driver for the suture anchor of FIG. 1;

FIG. 8 is a side elevation view of the driver shown in FIG. 7;

FIG. 9 is a top plan view of the driver shown in FIG. 8;

FIG. 10 is a cross sectional view of the driver of FIG. 9 taken along line 10′-10′ with anchor mounted therein; and

FIG. 11 is a perspective view of the driver with suture anchor mounted thereto.

### DETAILED DESCRIPTION OF THE PREFERRED EMBODIMENT

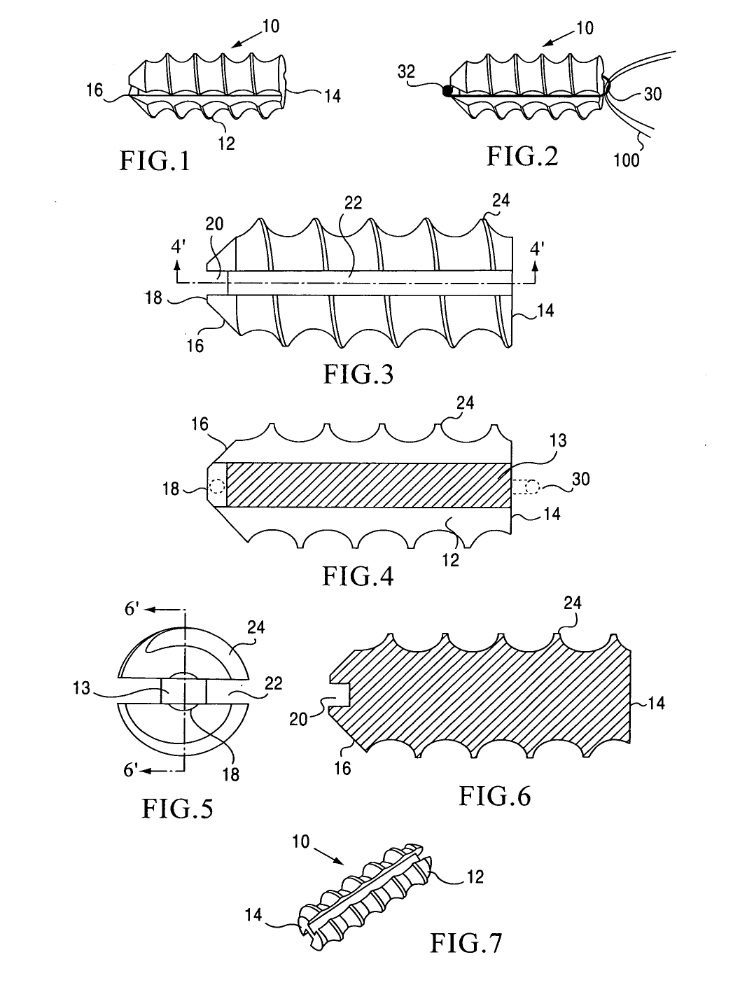

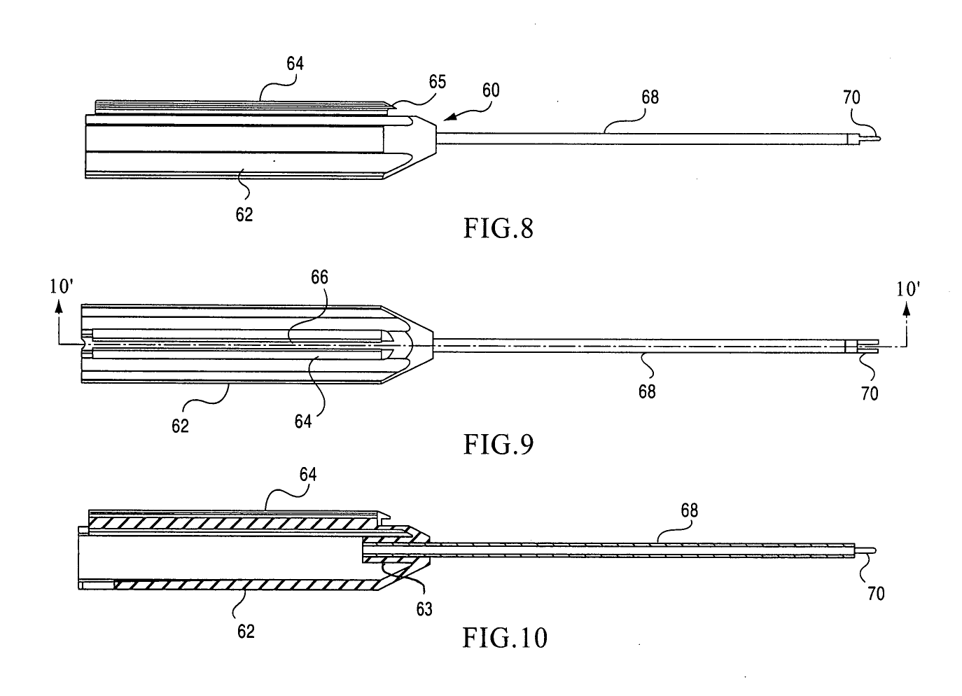

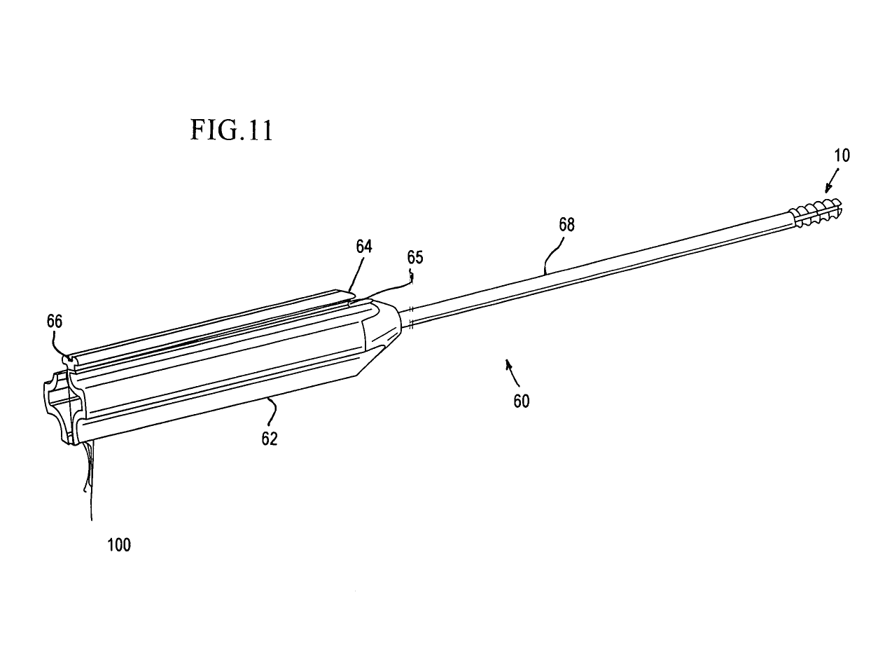

The preferred embodiment and the best mode of the invention as shown in FIGS. 1 through 11 is a suture anchor 10 with a cylindrical body 12 having a flat proximal end 14 and a tapered distal end 16 with a planar transverse tip 18 which is initially inserted into a bore cut in the bone mass (not shown). The distal end 16 tapers inward in about 45° from the center longitudinal axis of the suture anchor for self centering insertion and has a smooth flat end surface defining a connecting groove 20 which connects side grooves 22 and forms a seat for the suture loop 30. A helical thread 24 is cut in the cylindrical body 12. Preferably, the bone anchor is manufactured from human bone which is formed of mineralized cortical bone. Alternatively, the anchor body may be partially demineralized and alternately treated with bone morphogenic protein, hylauronic acid and a phosphate buffer for quicker bone formation.

It is also envisioned that the suture anchor may be manufactured from a biocompatible and bioresorbable material such as xenograft bone, polymeric or a biocompatible metal such as titanium or stainless steel.

The proximal end 14 of the suture anchor 10 is flat for minimum soft tissue impingement after insertion of the suture anchor 10 in the bone. Grooves 22 are cut into the opposing side surfaces of the cylindrical body 12 allowing the end bone anchor engaging members 70 of driver 60 to be easily and properly seated in the side grooves 22 of the suture anchor 10 to deliver driving torque to same. The width of each groove 22 is approximately 1.25 mm±0.05 mm and each groove 22 is positioned on opposite sides of the anchor body. An internal core structure 13 having a width ranging from 2.20 to 2.30 mm is formed after the grooves are cut which lends stability to the anchor body. The diameter of the suture anchor 10 preferably runs between 4.7 mm and 6.5 mm and the length from 10.0 mm to 15.0 mm with a preferred length of 15 mm. Threads 24 are cut in the body 10 in a helical pattern with a height of 0.90 mm and a pitch of 2.75 mm. The suture/driver channels or grooves 22 are longitudinally cut parallel to each other on opposite sides of the body 12 intersecting threads 24. If desired, any number of standard machine threads of appropriate size and thread configuration can be used. The channels or grooves 22 have a width greater than or equal to the diameter of the strand of suture loop 30 and a depth which is preferably at least twice the diameter of the strand of suture loop 30 extending into the anchor body. The suture loop 30 is preferably a #2 suture, a standard suture made of absorbable, synthetic absorbable or non-absorbable material which is tied in a knot 32 at the distal end of the suture anchor and fused to form a closed loop.

Sutures 100 such as ORTHOBRAID® are then inserted through the end of the loop 30 extending from the suture body at the distal end 14. The drive/suture holding grooves 22 are constructed so that the suture loop 30 will track in the grooves while the driver inserter tip members 70 are inserted in the grooves 22 to drive the anchor 10. The suture loop feature of the bone anchor allows easy suture loading and provides significant advantages over other threaded designs.

The suture anchor 10 is adapted for insertion into the distal end of a driver 60 which is shown in FIGS. 7 through 11. The driver 60 has a handle 62 constructed of plastic with one section of the handle forming a suture holder 64. This section defines a groove 65 around which the suture 100 can be wrapped and also defines upper suture guide groove 66. A stainless steel hollow shaft 68 is secured to the handle 62 in boss 63 and the distal tip of shaft 68 defines groove engaging members 70. The tip members 70 are in the form of two prongs, each of which extend into a respective groove 22 located on the side of the anchor body. The tip members 70 preferably have a width slightly less than the width of the grooves 22 so that when they are inserted into the grooves 22 they overlap the seated suture loop 30 so that driving torque can be applied to the suture anchor 10 groove walls via twisting of the driver handle 62 without damage to the suture loop 30. The suture anchor drive geometry is unique in that the suture loop 30 and driver tip members 70 use the same anchor groove 22.

In operation, the suture anchor 10 is mounted in the driver 60 with the tip members 70 mounted in the grooves 22 over the top of the suture loop 30. As the suture anchor is screwed into the bone bore, the bone mass surrounds the grooves 22 to hold the suture loop 30 within the respective groove 22 around the bone anchor. The suture anchor 10 is then seated in the bore previously drilled and tapped or punched into the bone with the proximal end substantially flush to the surface of the surrounding bone mass, the driver 60 having been backed off. The surgeon can then thread the sutures 100 through the anchor suture loop 30 and attach the suture 100 to the soft tissue and pull the soft tissue to the bone. Because the suture loop is a single piece of material, the failure strength is the suture line break strength rather than the pull out strength where two separate pieces of suture are used. Pull out of the anchor is also diminished because of the threaded seating in the bone mass.

In the foregoing description, the invention has been described with reference to a particular preferred embodiment, although it is to be understood that specific details as shown are merely illustrative, and the invention may be carried out in other ways without departing from the true spirit and scope of the following claims.

What is claimed is:

1. A sterile suture anchor comprising:
a substantially cylindrical body with an external screw thread extending along said cylindrical body,
a plurality of parallel longitudinal grooves are cut into said cylindrical body in the side of said body interrupting opposed portions of said screw thread;
a connecting groove is transversely cut through a distal end portion of said body connecting said plurality of longitudinal grooves, said longitudinal grooves and said connecting groove being dimensioned to hold at least one suture strand; and
a closed suture loop mounted to said cylindrical body in said longitudinal and connecting grooves.

2. A sterile suture anchor according to claim 1 wherein said longitudinal grooves run substantially the length of said cylindrical body.

3. A sterile suture anchor according to claim 1 wherein said suture loop is a single strand of suture with the ends knotted together to form a loop.

4. A sterile suture anchor according to claim 3 wherein said suture loop knot ends are fused.

5. A sterile suture anchor according to claim 1 wherein said cylindrical body has a tapered distal end and a flat tip which is transverse to the axis of the cylindrical body.

6. A sterile suture anchor according to claim 5 wherein said taper is about 45°.

7. A sterile suture anchor according to claim 1 wherein said longitudinal grooves are adapted to receive a portion of said suture loop so as to: recess said suture loop within said grooves and seat said suture loop so that said suture loop will not interfere with the receipt of a torque applying portion of a suture anchor driver.

8. A sterile suture anchor as claimed in claim 1 wherein said suture anchor is constructed of allograft bone.

9. A sterile suture anchor as claimed in claim 1 wherein said suture anchor is constructed of xenograft bone.

10. A sterile suture anchor as claimed in claim 1 wherein said suture anchor is constructed of plastic.

11. A sterile suture anchor as claimed in claim 1 wherein said suture anchor is constructed of metal.

12. A sterile suture anchor as claimed in claim 11 wherein said suture anchor metal is titanium.

13. A sterile biocompatible absorbable suture anchor comprising:
a substantially cylindrical body of allograft bone with a tapered distal end portion,
a screw thread cut in said cylindrical body,
a plurality of parallel longitudinal grooves cut into said cylindrical body interrupting opposed portions of said screw thread;
a suture loop groove cut in said tapered distal end portion engaging said plurality of longitudinal grooves, said suture loop groove being dimensioned to hold at least one suture; and
a closed suture loop member mounted in said longitudinal grooves and said suture loop groove.

14. A sterile suture anchor according to claim 13 wherein said cylindrical body tapered distal end is angled about 45°.

15. A sterile suture anchor according to claim 13 wherein said cylindrical body has a proximal end which is planar and is positioned about 90° in relation to a central axis of said cylindrical body.

16. A sterile suture anchor as claimed in claim 13 wherein said allograft bone is human cortical bone.

17. A sterile suture anchor comprising:
a cylindrical body with an angled distal end portion ending in a flat transverse tip; said cylindrical body having a planar proximal end which is substantially transverse to a central axis of said cylindrical body,
an exterior thread formed on said cylindrical body;
a plurality of parallel longitudinal grooves cut into said cylindrical body a depth greater than the diameter of a strand used in a suture loop;
a suture loop connecting groove cut through said distal end portion a depth greater than the diameter of a strand used in a suture loop engaging said plurality of longitudinal grooves, said suture loop connecting groove being dimensioned to hold at least one strand used in a suture loop; and
a closed suture loop of at least one strand mounted in said longitudinal grooves and said suture loop connecting groove.

18. A sterile suture anchor and drive kit comprising:
a suture anchor with a cylindrical body portion with a generally tapered distal end portion;
a screw thread extending along said cylindrical body portion;
a plurality of parallel longitudinal grooves cut into said cylindrical body interrupting opposed portions of said screw thread;
a connecting groove cut through said distal end portion engaging said plurality of longitudinal grooves;
a closed suture loop mounted in said longitudinal grooves and said connecting groove; and
a driver adapted to be mounted to said suture anchor, said driver comprising a handle, a tube mounted to said handle, said tube being provided with outwardly extending drive tip members adapted to be inserted into said suture anchor longitudinal grooves and engage groove side walls of said suture anchor to apply driving torque to said suture anchor.

19. A sterile suture anchor and drive kit as claimed in claim 18 wherein said handle defines a suture holding recess.

20. A sterile suture anchor and drive kit as claimed in claim 18 wherein said handle defines a suture guide channel.

21. A sterile suture anchor and drive kit as claimed in claim 18 wherein said drive tip members are two opposed prongs, each prong having a width less than the width of said longitudinal grooves.
